(19) United States
(12) Patent Application Publication
(10) Pub. No.: US 2012/0077869 A1
(43) Pub. Date: Mar. 29, 2012

(54) METHOD AND COMPOSITION USING A DUAL SPECIFICITY PROTEIN TYROSINE PHOSPHATASE AS AN ANTIMALARIAL DRUG TARGET

(75) Inventors: John H. Adams, Tampa, FL (US); Bharath Balu, Harrisonburg, VA (US); Steven P. Maher, Tampa, FL (US); Christopher O. Campbell, Tampa, FL (US); Roman Manetsch, Tampa, FL (US)

(73) Assignee: UNIVERSITY OF SOUTH FLORIDA, Tampa, FL (US)

(21) Appl. No.: 13/237,525

(22) Filed: Sep. 20, 2011

Related U.S. Application Data
(63) Continuation of application No. PCT/US2010/028152, filed on Mar. 22, 2010.
(60) Provisional application No. 61/162,009, filed on Mar. 20, 2009.

(51) Int. Cl. A61K 31/5377; A61K 31/122; A61K 31/7088; A61K 31/403; A61P 33/06; A61K 31/655; A61K 31/4365

(52) U.S. Cl. 514/44 R; 514/235.2; 514/150; 514/681; 514/301; 514/411

(57) ABSTRACT

Phosphotyrosine phosphatase (PTP) encoded by PF13_0027 is a desirable drug target for the human malaria parasite Plasmodium falciparum. This PTP is critical for intraerythrocytic parasite development and invasion of erythrocytes by malaria merozoites. Mutation of the PF13_0027 gene or blocking expression of PTP function to create a PTP-null parasite severely attenuates the malaria parasite's ability to survive, making the PTP-null parasite suitable as an attenuated blood-stage parasite vaccine.

### CROSS REFERENCE TO RELATED APPLICATIONS

This application is a continuation of prior filed International Application, Serial Number PCT/US2010/028152 filed Mar. 22, 2010, which claims priority to currently pending U.S. Provisional Patent Application No. 61/162,009, filed Mar. 20, 2009, the contents of which are herein incorporated by reference.

### STATEMENT OF GOVERNMENT INTEREST

This invention was made with Government support under Grant Nos. R01 AI033656 and R21 AI070888 awarded by the National Institute of Allergy and Infectious Disease (NIAID). The Government has certain rights in the invention.

### FIELD OF INVENTION

This invention relates to the treatment of malaria. Specifically, this invention relates to the discovery of a novel drug target for the treatment of malaria.

### BACKGROUND OF THE INVENTION

Malaria is a devastating disease that kills 2-3 million people and is responsible for 300-500 million clinical infections each year. There is no vaccine available to prevent infection and there is widespread resistance to anti-malarial drugs, necessitating a continued need for new drug discovery and development. Malaria is caused by a protozoa in the genus Plasmodium. Four species cause human malaria: P. vivax, P. malariae, P. ovale, and P. falciparum. Of the four Plasmodium species that cause malaria, Plasmodium falciparum is responsible for much of the mortality associated with the disease primarily due to lethal injections in young children in sub-Sarahan Africa. The Plasmodium pathogens are generally transmitted to humans by mosquitos but can also be transmitted by infected blood or needles. Once the sporozoites enter the bloodstream, they localize in the liver cells and one to two weeks later, the infected liver cells rupture and release mature pathogens or merozoites. These merozoites then begin the erythrocytic phase of malaria by attaching to and invading erythrocytes.

Phosphorylation Cascades in Plasmodium

Protein phosphorylation plays an important role in eukaryotic cell development by regulating the activity of various cell cycle checkpoints—kinases add phosphate groups and phosphatases remove them. Knockouts of kinase function can alter proliferation and response to external stimuli, which in the case of Plasmodium was shown as failure to develop through the sexual cycle. Kinases have become the focus of several studies, because of their deduced importance for Plasmodium development. Much of the focus is on their involvement in cascades regulating the progression from gametocyte to the formation of the zygote and oocyst in the mosquito midgut. There are no comparable studies with blood stage parasites because genetic methods are limited and the haploid genome does not tolerate deleterious gene KO's.

Calcium dependant protein kinases (CDPKs) make up a family of serine/threonine kinases found only in protozoa and plants and are distinct from all other animal protein kinases (Schliker, C., Mogk, A., & Bukau, B. (2004). Gamete Interruptus: A Novel Calcium-Dependant Kinase is Essential for Malaria Sexual Reproduction. Cell, 419-420). The P. falciparum genome encodes 6-7 CDPKs; those that are developmentally regulated by Ca2+ signaling are best characterized in the parasite sexual stages that develop within the mosquito midgut (Billker, O., Dechamps, S., Tewari, R., Wenig, G., Franke-Fayard, B., & Brinkmann, V. (2004). Calcium and a Calcium Dependant Protein Kinase Regulate Gamete Formation and Mosquito Transmission in a Malaria Parasite. Cell, 503-514). CPDK4 specifically is one of the kinases responsible for the transduction of the Ca2+ signals within the parasite required for differentiation of blood-stage gametocytes to mosquito stage gametes. Signal transduction resulting from an increase in the exogenous levels of Ca2+ promotes the cycle of mitosis that produces the microgametes (Arai, M., Billker, O., Morris, H. R., Panico, M., Delcroix, M., Dixon, D., et al. (2001). Both Mosquito-derived Xanthurenic acid and a Host blood-derived gametogenesis of Plasmodium in the midgut of the mosquito. Molecular and Biochemical Parasitology, 17-24). When CDPK4 is knocked out, it results in zero oocyst, leading to the understanding that its expression is essential (Muhia, D. K., Swales, C. A., Deng, W., Kelly, J. M., & Baker, D. A. (2001). The Gametocyte-activating Factor Xanthurenic acid Stimulates an increase in Membrane-associated guanylyl cyclase activity in the Human Malaria parasite Plasmodium falciparum. Molecular Microbiolgy, 553-560).

The P. falciparum cell cycle seems likely to be driven by a sequential activation of cyclin dependant kinases similar to higher eukaryotes, if the processes observed for sexual cycle control reflect the general regulatory pattern (Bonnet, J., Mayonove, P., & C., M. M. (2008). Differential Phosphorylation of Cdc25C Phosphatase in Mitosis. Biochemical and Biophysical Research Communications, 483-488). CDC25 typically acts as a cell cycle regulator by activating the cyclin dependant kinases (CDKs) through dephosphorylation at the G2-M transition (Rudolph, J. (2007). Cdc25 Phosphatases: Structure, Specificity and Mechanism. Biochemistry, 35953604; Contour-Galcera, M.-O., Sidhu, A., Prevost, G., Bigg, D., & Ducommun, B. (2007). What's new on CDC25 Phosphatase Inhibitors. Pharmacology and Therapeutics, 115 (1), 1-12).

In humans there are three CDC25 phosphatases that dephosphoryate the T/Y residues in order to trigger activation of CDK activity (Rudolph, 2007). Intertwined in these activities are more poorly characterized mitogen-activated protein kinases (MAPKs) that are also involved in the control of cytokinesis and motility during the gamete formation in the malaria parasite (Tewari, R., Dorin, D., Moon, R., Doerig, C., & Billker, O. (2005). An Atypical Mitogen-activated protein kinase controls cytokinesis and flagellar motility during male gamete formation in the malaria parasite. Molecular Microbiolgy, 1263-1263). Although their precise functions are still the topic of many questions, the kinome of P. berghei possesses two MAPKs, MAP-1 and MAP-2 with different roles in parasite survival (Dorin-Semblat, D., Quashie, N., Halbert, J., Sicard, A., Doerig, C., Peat, E., et al. (2007). Functional characterization of both MAP kinases of the human malaria parasite Plasmodium falciparum by reverse genetics. Molecular Microbiolgy, 1170-1180).

Phosphatases are required for protein dephosphorylation, which control the actions of protein kinases, since unregulated kinase activity is detrimental to cell viability. There are two main functional groups of protein phosphatases; protein tyrosine phophatases (PTP) which are typically membrane bound and protein serine/threonine phosphatases, which are located in the cytoplasm (Lindenthal, C., & Klinkert, M. (2002). Identification and biochemical characterization of a protein phosphatase 5 homologue from Plasmodium falciparum. Molecular and Biochemical Parasitology, 257268; Kumar, R., Adams, B., Oldenburg, A., Musiyenko, & Barik, S. (2002). Characterization and expression of a PP1 serine/threonine protein phosphatase (PfPP1) from the malaria parasite, Plasmodium falciparum: demonstration of its essential role using RNA interference. Malaria Journal). Members of the specific phosphatase families have high sequence conservation within the active site. The participation of phosphatases in regulation of cell cycle, protein synthesis, carbohydrate metabolism, and transcription in eukaryotic cells underscores their importance to survival (Kumar et al., 2002).

The post-genome era has shown a progression of functional genomics studies on P. falciparum that has provided valuable information about parasite biology. More than 50% of the genes in Plasmodium genomes code for hypothetical proteins with limited homology to model organisms thus high throughput methods for identification of gene functions are necessary to understand parasite biology and develop effective disease control strategies.

Symptoms of the disease include high fever, chills, headaches, anemia and splenomegaly. There are a limited number of drugs that can treat malaria. A continuing rise in parasite drug-resistance has hindered malaria control strategies and resulted in an increased number of deaths in the last few years.

As stated above, malaria is the result of infection with apicomplexan parasites of the genus Plasmodium and is transmitted to humans by Anopheles species. The life cycle of Plasmodium is a complex process dependant on precise stage specific gene expression in different hosts and multiple cell types. Genes are expressed in a continuous cascade-like pattern in correlation with phases of development allowing the smooth progression of the parasite life cycle. The inventors' studies demonstrate that knocking out a gene for a conserved phosphotyrosine phosphatase (PTP), which has structural similarities to CDC25, results in an altered cell cycle length and a drastically impaired blood-stage development process. Multiple significant defects identified in the mutant parasites illustrate the PTP can be used as a drug target or null parasites as a whole-parasite attenuated vaccine. The burden of malaria in endemic regions exerts a considerable burden both economically and socially, making it a serious public health concern.

### SUMMARY OF INVENTION

The present invention combines novel genetic and chemistry approaches to aid development of drug chemotypes with novel effector mechanisms to target a vulnerable pathway in P. falciparum. One embodiment of the present invention is a method of treating malaria comprising contacting a cell infected with a Plasmodium species with a therapeutically effective amount of a phosphatase inhibitor. The cell is preferably a red blood cell and the phosphatase inhibitor can be a CDC25 phosphatase inhibitor or a dual specificity protein tyrosine phosphatase inhibitor. The phosphatase inhibitor preferably targets a gene in a Plasmodium species having a nucleic acid sequence having homology to SEQ ID NO:2. The Plasmodium species is P. falciparum or P. vivax.

Another embodiment includes a method of preventing malaria through regulating the cell cycle of a Plasmodium species by inhibiting the expression of the P13_{—}0027 gene. The Plasmodium species is P. falciparum or P. vivax. The P13_{—}0027 gene can be inhibited by a phosphatase inhibitor. The phosphatase inhibitor can be a CDC25 phosphatase inhibitor or a dual specificity protein tyrosine phosphatase inhibitor. Alternatively, the expression of P13_{—}0027 can be inhibited by the insertion of a single transposon at a TTAA sequence in the open reading frame.

A further embodiment includes a method of preventing malaria through administering a therapeutically effective amount of a PTP-null Plasmodium species and a pharmaceutically acceptable carrier. The PTP-null Plasmodium species can have the insertion of a single transposon at a TTAA sequence in the open reading frame.

Another embodiment includes a pharmaceutical composition for preventing malaria comprising a PTP-null Plasmodium species and a pharmaceutically acceptable carrier with the PTP-null Plasmodium species having the insertion of a single transposon at a TTAA sequence in the open reading frame.

### BRIEF DESCRIPTION OF THE DRAWINGS

For a fuller understanding of the invention, reference should be made to the following detailed description, taken in connection with the accompanying drawings, in which:

FIG. 1 is a graph depicting the growth rate analysis of PF13_{—}0027 mutant (ΔPF13_{—}0027) versus wild-type Plasmodium falciparum (NF54). Growth rates were calculated from asynchronous parasite cultures quantified by flow cytometry at 24-hour intervals for 7 days. Doubling time for the mutant parasite was significantly longer at 21.01 hours versus 18.39 hours for NF54. Parasite fold change is depicted on the y-axis and time in hours is depicted on the x-axis.

FIG. 2A is an illustration of the insertion site location of the piggyBac element carrying the drug resistance cassette (hdhfr) into the ΔPF13_{—}0027 mutant at the PF13_{—}0027 locus. It was located in the N-terminal region of the CDS.

FIG. 2B is an image of an agarose gel in which wild-type and the ΔPF13_{—}0027 mutant C9 nucleic acid products were analyzed. As shown in the image, the insertion of the piggyBac element carrying the drug resistance cassette abrogated normal expression of the gene as no mRNA from this locus was detected in the developing mutant parasite. This result indicates the disruption of ΔPF13_{—}0027 results in a loss-of-function mutant phenotype.

FIG. 2C is an image illustrating that quantitative RT-PCR of wild-type NF54 parasite populations determined the normal transcription of PF13_{—}0027 to peak in late trophozoite stages, which is a pre-S phase time of development in P. falciparum, just prior to onset of DNA synthesis and serial nuclear division as in the Giemsa-stained blood smears representative of the typical development pattern.

FIG. 3A is an image depicting the multiple sequence alignment of the conserved Rhodanese domain (RHOD) identified in PF13_{—}0027 (SEQ ID NO: 3) compared to RHOD present in genes of other species.

FIG. 3B is an image illustrating that the analysis of phylogenetic relatedness of these RHOD demonstrates that the RHOD of PF13_{—}0027 and its orthologs in other Plasmodium species are distinct from the closest related genes of humans and other mammals.

FIG. 3C is an image depicting the multiple sequence alignment of the conserved dual specificity phosphatase domain (PTP) identified in PF13_{—}0027 (SEQ ID NO: 4) compared to PTP present in genes of other species. Arrows identify residues conserved among PTP relating to catalytic function reveal PF13_{—}0027 is unique.

FIG. 3D is an image illustrating that the analysis of phylogenetic relatedness of these PTP demonstrates that the PTP of PF13_{—}0027 and its orthologs in other Plasmodium species are distinct from the closest related genes of humans and other mammals. The presence of these two domains (RHOD, PTP) together in PF13_{—}0027 identify its gene product as a unique dual specificity protein tyrosine phosphatase associated with regulating function of mitogen-activated kinases of P. falciparum.

FIG. 4A is a graph depicting the detailed growth curve analysis of highly synchronized ΔPF13_{—}0027 knockout mutant parasites (KO) parasites compared to wild-type NF54 (WT). The three main stages of parasite development (rings, trophozoites, schizonts) were quantified by flow cytometry at 2 hour intervals over three generations of development (150 hours). It was determined that the longer generational time of the mutant was due to a delay in the transition from pre-S to S phase of development, which corresponds to the period of maximal expression in NF54 parasites and indicating a defect in the function of a CDC25-like PTP phosphatase activation mechanism associated with loss of the PF13_{—}0027 gene product.

FIG. 4B is an image of the design of the intact PF13_{—}0027 gene incorporated into the piggyBac transposon. The intact PF13_{—}0027 gene was placed on a piggyBac transposon with a bsd drug selection marker and reinserted into the genome of the mutant KO parasite to create a genetic rescue and restore or complement the mutation.

FIG. 4C is a graph illustrating the percent fold change of wild-type of the mutant parasite line before and after genetic rescue. As shown by the graph, complementation of the intact PF13_{—}0027 gene into the mutant parasite restored the normal growth phenotype at 100% of the wild-type NF54 parasite.

FIGS. 5A and B are images depicting the ultrastructure of the wild-type parasite (a) was compared to the mutant PF13_{—}0027 parasite clone C9 (b) identified significant differences at the end of schizont development. The parasitophorous vacuole membrane was prematurely lost in the mutant parasite leading to defective maturation of developing merozoites and atypical egress. Increased abundance of knobs in the mutant parasite indicated increase levels of exported proteins.

FIG. 5C is an image depicting that the defects in schizont development and merozoite maturation resulted in release of largely non viable merozoites that accumulated in the supernatant of in vitro cultures.

FIG. 5D are a series of images showing that the merozoites are capable of initiating early stages of the invasion process but were not able to complete the invasion process (100 or 100 observed).

FIG. 6 is an image of the nucleotide sequence of the P13_{—}0027 KO gene (SEQ ID NO: 2). The amino acid sequence of the polypeptide (SEQ ID NO: 1) is shown below the nucleic acid sequence and is the same as that depicted in FIG. 7. The TTAA area highlighted in black represents the insertion site at nucleotides 199-202 of the PF13_{—}0027 open reading frame. Insertion at this site disrupts the coding sequence and abrogates mRNA production as shown in FIG. 2B. The dark grey shaded area represents the rhodanese domain. The light grey shaded area represents the phosphatase domain. The medium grey area represents the transmembrane domain. Two hydrophobic sequences are present and may serve as transmembrane domains (medium grey).

FIG. 7 is an image depicting the amino acid sequence of PF13-0027 (SEQ ID NO: 1) CDS showing the rhodanese domain (light grey) and dual specificity phosphatase domain (medium grey). The substitution of the critical cysteine residue of the rhodanese domain is shown at position 144. This residue substitution inactivates the rhodanese domain. The critical residues of the catalytic site in the phosphatase domain are D345, C383, and 1398.

FIGS. 8A and B are homology models of the (a) RHOD and (b) PTP of PF13_{—}0027 created based on the best fit with their nearest neighbors, 1c25 and Pyst1, respectively.

FIG. 8C identifies signature motif residues of a consensus PTP at positions 345, 382, 398 that should be amino acids Aspartic acid (D), Cysteine (C), and Arginine (R) are conserved except an Isoleucine (I) that replaces the R, identifying catalytic site of the PF13_{—}0027 product is unique.

FIG. 8D is a refined homology model that displays the loop insertion that further modifies the PTP catalytic site of the PF13_{—}0027 product and is present only in orthologs of other Plasmodium species.

FIG. 9A is an image of immunoblot detection. The PF13_{—}0027 transgene created to express the malarial phosphatase with a C-terminal HA tag was introduced into the KO mutant parasite clone and expression was confirmed by immunoblot detection of the HA tag. Immunoprecipitation of the transgene product by an anti-HA antibody identified multiple proteins interacting with the parasite phosphatase.

FIG. 9B is an image illustrating multiple peptide fragments identified for elongation factor 1a and protein 14-3-3, which are typically associated with CDC25 and regulate its function related to initiating the G1-S transition. Interaction with actin identifies an activity associated with the altered invasion phenotype demonstrated for the ΔPF13_{—}0027 mutant as shown in FIG. 5D.

FIG. 10 is an image depicting known phosphatase inhibitors. Minimum inhibitory concentration (MIC) assays were carried out in vitro to get a baseline level of sensitivity for these drugs in P. falciparum. Current phosphatase inhibitors were used to test their effects on the growth of P. falciparum. These phosphatase inhibitors are known to act against CDC25 types of phosphatases and dual specificity protein tyrosine phsophatases such as shp1 and shp2. Effective inhibition established from this in vitro assay indicates P. falciparum shares functional homologs of these phosphatases.

### DETAILED DESCRIPTION

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

The “therapeutically effective amount” for purposes herein is used to denote the amount of a composition needed to be administered in order to effectuate a beneficial result or change, whether that change is an improvement such as stopping or reversing the degeneration of a disease or condition, reducing a deficit or improving a response, or a complete cure of the disease or condition treated. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of the animal and the route of administration. The therapeutically effective amount of the composition can be administered to any type of cell known to be susceptible to infection or damage from malaria, including but not limited to, erythrocytes and hepatocytes.

“Administration” or “administering” is used to describe the process in which a compound or combination of compounds of the present invention are delivered to a patient. The composition may be administered in various ways including parenteral (referring to intravenous and intraarterial and other appropriate parenteral routes), intratheceal, intraventricular, among others which term allows the composition of the subject invention to migrate to the ultimate site where needed. Each of these conditions may be readily treated using other administration routes of compound or any combination of compounds thereof to treat a disorder or condition.

Determining the Degree of Sequence Identity

The invention provides polypeptides having at least 90%, 91%, 92%, 93%, 94%, 95%, 96%, 97%, 98%, 99% or more sequence identity to SEQ ID NO: 1. These polypeptides can be generated from the nucleic acid sequence having at least 90%, 91%, 92%, 93%, 94%, 95%, 96%, 97%, 98%, 99% or more sequence identity to SEQ ID NO:2. The sequence identities can be determined by analysis with a sequence comparison algorithm or by a visual inspection.

Protein and/or nucleic acid sequence identities (homologies) may be evaluated using any of the variety of sequence comparison algorithms and programs known in the art. The extent of sequence identity (homology) may be determined using any computer program and associated parameters, such as those described in US 2004/0072228 A1, such as BLAST 2.2.2. or FASTA version 3.0t78, with the default parameters.

The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, contiguous residues ranging anywhere from 1 to the full length of an exemplary polypeptide or nucleic acid sequence of the invention, e.g., SEQ ID NO:1, SEQ ID NO:2, are compared to a reference sequence of the same number of contiguous positions after the two sequences are optimally aligned.

The phrase “substantially identical” in the context of two nucleic acids or polypeptides, can refer to two or more sequences that have, e.g., at least about at least 90%, 91%, 92%, 93%, 94%, 95%, 96%, 97%, 98%, 99% or more sequence identity or more nucleotide or amino acid residue (sequence) identity, when compared and aligned for maximum correspondence, as measured using one any known sequence comparison algorithm, as discussed in detail below, or by visual inspection. Nucleic acid sequences of the invention can be substantially identical over the entire length of a polypeptide coding region.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin EW [1995] Easton Pa., Mack Publishing Company, 19^{th} ed.) describes formulations which can be used in connection with the subject invention.

The term “PTP-null Plasmodium species” as used herein refers to the mutant P13_{—}0027 in which a single transposon is inserted into the open reading frame of the gene at a TTAA sequence to inactivate the PTP domain of the protein. This species can be used as a vaccine adjuvant. The PTP-null Plasmodium species is also known as “C9” and “ΔPF13_{—}0027”

Malaria caused by Plasmodium falciparum is a devastating disease responsible for more than 1 million deaths and 300-500 million clinical illnesses annually. Clinical disease results from cyclical asexual development of this protozoan parasite in the blood by a seemingly rigid cascade of gene expression insensitive. Many of the anti-malarial drugs used to control malaria are rapidly losing their efficacy due to the adaptations of the parasite and the common chemical nature and targets of many current drugs. There is an urgent need to identify new targets for novel combination therapies to prevent emergence of resistance and prolong effectiveness of current drugs. Studies deciphering the unique metabolic processes of Plasmodium biology play an important role to identify novel that can be evaluated as new targets for therapeutic interventions.

It is widely believed that the observed cyclical pattern of malaria parasites is ‘hard wired’ into the genome, which contrasts most eukaryotic organisms that highly regulate cell cycle development. As stated above, typically, kinases and phosphatases regulate the critical cellular processes of cell cycle division through protein phosphorylation and dephosphorylation. Protein phosphatases dephosphorylate proteins, often acting as checkpoints during a eukaryotic cell cycle, while kinases often initiate progression through the cycle by phosphorylation. Maturation of mature sexual stages of malaria parasites is regulated by a series of kinases and there is evidence that processes of protein phosphorylation are also vital to the efficient regulation and survival of P. falciparum blood-stage development. Sequential activation of cyclin dependant kinases was identified as important for cell cycle progression of P. falciparum (Bonnet et al., 2008). CDC25 typically is an essential regulator of the cell cycle that works by activating the cyclin dependant kinases (CDKs) through dephosphorylation at the G1/S and G2/M transitions (Rudolph, 2007; Contour-Galcera et al, 2007). In humans, there are three CDC25, which dephosphoryate the Thr and Tyr residues in order to trigger activation of CDK/cyclin activity (Rudolph, 2007). Since unregulated kinase activity is also detrimental to cell viability, protein phosphatases (PPs) are required for controlling the actions of protein kinases by dephosphorylation.

Protein phosphatases are required for the dephosphorylation of proteins reversing and controlling the actions of protein kinases, since unregulated kinase activity is also detrimental to cell viability. There are two main functional groups of protein phosphatases; protein tyrosine phophatases (PTP) which are typically membrane bound and protein serine/threonine phosphatases (PP) which are located in the cytoplasm (Lindenthal & Klinkert, 2002; Kumar et al., 2002). The members of the specific phosphatase families have high sequence conservation within the active site. The participation of PPs in regulation of cell cycle progression, protein synthesis, carbohydrate metabolism, transcription and neuronal signaling in eukaryotic cells underscores their importance to survival (Kumar et al., 2002). Similar to the kinases that phosphorylate serine and tyrosine residues on substrates, the PPs carry out the inverse reaction, removing phosphates from those residues. Within the PPs, there are two distinct families; the Mg²⁺-dependant phosphatases (PPM) and Mg²⁺-independent phosphatases (PPP) (Lindenthal & Klinkert, 2002). The PPs can be further categorized into more specific groups, such as; PP1, PP2A, PP2B and PP2C (which is a PPM). Relatively little functionality is available for the roles of phosphatases in malaria parasite biology.

Random mutagenesis is an effective tool to identify genes associated with specific phenotypes. The inventors have discovered a CDC25-like phosphatase that is important for transition from G1 into S/G2/M phase of P. falciparum blood-stages development. This discovery came from isolation of a slow growing mutant parasite carrying a knockout of a conserved hypothetical protein of unknown function (PF13_{—}0027; SEQ ID NO: 1) created by random transposon-based insertion mutagenesis (piggyBac). Analysis of the disrupted open reading frame identified tandem phosphatase domains, a rhodanese domain followed by dual specificity protein tyrosine phosphatase II (PTP). The inventors discovered PTP to be an essential dual specificity phosphatase with CDC25-like phosphatase properties in cell cycle regulation of mitotic development stage of Plasmodium.

CDC25 in normal eukaryotic cells has a critical role regulating entry into mitosis and S phase. A knockout of PF13_{—}0027 delays entry through S/G2/M phase significantly extending parasite development time. Phosphorylation by parasite-specific protein kinases regulate essential steps for progression through Plasmodium sexual stage development. The human CDC25s are well characterized and are intensively studied as targets of anti-cancer drug therapy.

The inventors developed a forward genetic approach to study the genome of P. falciparum, which uses a random insertional mutagenesis method with the transposable element, piggyBac. This innovative technology was applied to screen for genes important for survival of the parasite during blood-stage development. This transposon-based methodology can be used to effectively create attenuated parasites.

PF13_{—}0027

The PF13_{—}0027 protein was examined as a potential new drug target in P. falciparum. Although not annotated as a CDC25 ortholog, this conserved gene has a rhodanese domain and a dual specificity phosphotyrosine protein phosphatase (PTP) domain similar to a CDC25, which are essential for survival in other eukaryotes, and appears to be involved in regulating cell cycle. The inventors have discovered that the PF13_{—}0027 PTP product is functionally equivalent to CDC25 phosphatase and has significant roles in the development of the parasite. Based upon the vital role for this protein, this putative CDC25-like P. falciparum phosphatase is an attractive new anti-malarial drug target since cascades involving protein phosphorylation are likely interdependent for successful development of Plasmodium parasites.

piggyBac

The piggyBac transposition system provides a powerful tool for knocking out genes in P. falciparum as well as for rapid stable integration of transgenes for expression analysis (Balu, B., Shoue, D., Fraser, M., & Adams, J. (2005). High-efficiency transformation of Plasmodium falciparum by the Lepidopteran transposable element piggyBac. 16391-16396). The ability to perform transposon-mediated mutagenesis in P. falciparum provides a sound platform to carry out several genetic analyses not previously available for Plasmodium. Transgene analysis is especially valuable when assessing the mutant genotypes to rescue the wild-type phenotype in the null parasite line. piggyBac is a “cut and paste” transposon that inserts into TTAA target sequences in the presence of a piggyBac transposase. (Balu, B. et al., piggyback is an effective tool for functional analysis of the Plasmodium falciparum genome (2009) BMC Microbiology; 9:83) piggyBac's insertionpreference for transcription units enhances its efficacy in large-scale mutagenesis studies to identify gene functions.

The inventors have discovered a novel genetically validated drug target during a whole-genome random mutagenesis screen. PF13_{—}0027 PTP has been genetically validated as a desirable drug target in P. falciparum, is conserved in P. vivax, and is a transmission blocking and prophylactic drug target. Currently there are no PTP inhibitors in development as anti-malarial drugs. PTP inhibitors must be highly charged to effectively interact with the phosphatase catalytic site limiting membrane permeability.

There is an abundance of information regarding human phosphatases, phosphatase inhibitors, and FDA approved anti-phosphatase drugs which allows the identification of lead compounds that are specifically effective against the parasite PTP. Several defined phenotypes have been defined through gene knockout analysis that can be used to evaluate drug effects during the discovery and optimization process.

Methods

Bioinformatics Analysis of Catalytic Domains, Conserved Residues and Homology Modeling

The deduced PF13-0027 protein sequence (SEQ ID NO:1) was compared to the phosphatases identified in Apicomplexa, including other Plasmodium spp. Often these parasites have numerous INDELS so this analysis included conserved domains and motifs present in phosphatases of humans and yeast. Only regions common to all the sequences from the respective genes were used for this analysis. Multiple alignments were carried out through the generation of a multiple sequence alignment, using MacVector® 10.0.1 (Accelerys) and using the ClustalW Multiple alignment editor. Cluster trees constructed using the neighbor-joining method with 1000 bootstrap determined phylogenetic relatedness. By aligning the functional domains of the other phosphatases with PF13_{—}0027 the inventors determined the type of the phosphatase functional domains of the sequences that had the greatest level of identity to PF13_{—}0027. The significant biological information available about the phosphatases of humans, other mammalian species and yeast was important for functional classification of PF13_{—}0027. Since the PF13-0027 protein (SEQ ID NO: 1) is considered a possible drug target, a comparison of similarity to the functional domains of the human phosphatases was carried out.

Using the crystal structure with the greatest identity to PF13_{—}0027 (SEQ ID NO: 1) in the Protein Data Bank (1 mkp), a homology model was developed using Swiss Model. Using the Swiss-Model software, a 3D model was generated using the selected template. The theoretical model of PF13_{—}0027 allowed the comparison of the structure and location of the functional residues and domains to the template model using Pymol molecular visualization software (Schrödinger). The positions of critical residues were also determined from this analysis.

Transfection and Identification of Insertion Sites

The piggyBac plasmids used for transfections were derived from previously reported plasmids pXL-BACII-DHFR and pHTH (Balu et al., 2005). pLBacII-HDH-eGFP used to create this mutation has a 200 bp region of 5 eba-175 that was amplified from the P. falciparum genome and cloned into pLBacII-HDH-GFP as a ClaI/ApaI fragment. The selectable marker hDHFR, which is commonly used for transformation of malaria parasites, was used to confer resistance to the anti-malarial compound WR99210 (Jacobus Pharmaceutical, Princeton, N.J.). A GFP C-terminal fusion tag was added for sorting and other applications. A helper plasmid pHTH expressed the piggyBac transposase in P. falciparum blood-stage parasites using the regulatory elements of P. falciparum hsp86. To minimize its size, the helper plasmid included no selectable marker. Transfections were performed using red blood cells as described previously (Balu et al., 2005). Briefly, transfection of P. falciparum NF54 was achieved by parasite invasion of RBCs ‘preloaded’ with plasmid DNA. Preloaded erythrocytes were washed with culture media and used immediately or stored at 4° C. To ensure parasite invasion of only plasmid-loaded RBCs, mature blood-stage parasites were purified on a MACS magnetic column (Miltenyi Biotec); 1 million purified parasites were added to erythrocytes loaded with 100 ug of the transposon plasmid and 50 mg of the transposase plasmid to start a 5 ml parasite culture. Drug selection was initiated at 48 hours post-transfection with 2.5 nM of WR99210 added to the culture and the parasites were maintained in drug for 8 days until parasites were first detected in Giemsa-stained smears. Individual mutant clones were obtained by limiting dilution of parasites post-drug selection.

The piggyBac insertion sites in the transformed parasites were identified from inverse PCR-amplified fragments. Genomic DNA (2 mg) extracted from transformed parasites was digested with 10 units of either Dra I or Rsa I and used in inverse PCR. The amplified PCR products were sequenced with primers in piggyBac inverted terminal repeats and analyzed using MacVector® 11 (Accelerys) Insertion sites were identified by performing BLAST searches using NCBI and PlasmoDB databases. Primers created to the flanking sequences confirmed the location and orientation of the piggyBac insert into PF13_{—}0027.

Flow Cytometry and Estimation of Doubling Times

Growth assays were performed by maintaining asynchronous cultures of P. falciparum wild-type and mutant clones at parasitemias 0.5-2% in 96-well plates by diluting every 48 hrs (Maher & Balu, unpublished data). Parasite cultures were plated in triplicate for each time point and samples were taken every 24 hrs for 7 days and fixed in 0.05% glutaraldehyde after removal of culture medium. Flow cytometry was used to estimate parasitemia by staining parasites with ethidium bromide and analyzed using the FACSCanto™ flowcytometry system (Becton, Dickinson and Company) in a high throughput format. A total of 20,000 cells were counted for each sample. The data were analyzed using FACSDIVA™ software (Becton, Dickinson and Company). Growth rate analyses were performed using SAS (9.1). The total number of parasites (y) (parasitemia X dilution factor), was plotted against time (x) and fitted to the exponential growth curve [y=m0*e^{(ln2*x/D)}] (where, D is the intrinsic parasite doubling time and m0 is the theoretical parasite number at time 0). To compare directly the growth rate of parasite clones with slightly different starting parasitemias, the—fold increase of the parasite number, normalized to have a single theoretical parasite for each culture at time 0, was used for graphing the growth curve. One hundred parameter initiation values ranging from 5 to 105 were tested and the best converging model with the smallest Sum Square of Error (SSE) was chosen for estimation of doubling time.

Phenotype Rescue by Complementation

The full-length PF13_{—}0027 was inserted the genome of the C9 mutant using a piggyBac element also carrying the BSD gene. The transformation protocol used was similar to what was described above for generating the mutant with the hDHFR knockout vector. The PF13_{—}0027 transgene restored normal growth time and growth pattern thus rescuing the wild-type phenotype.

### Preparation of the pGEX-2T with PF13_{—}0027 Phosphatase Domain Insert for Protein Expression

Primers for the PF13_{—}0027 phosphatase domain were designed to produce an insert with 5′ BamHI and 3′ EcoRI restriction sites for cloning into the expression vector PGEX-2T. The forward primer (aaaggatccATGTATATAAATTATCCTATAAAAATGTTTGATAAC; SEQ ID NO: 35) was designed with a melting temperature of 56.9° C. and the reverse primer (tttgaattcCTTAATTAGGGATTGATAGAAACTTTC; SEQ ID NO: 36) with an annealing temperature of 56.2° C.

The phosphatase domain was amplified by PCR using High Fidelity® Platinum Taq polymerase (Invitrogen cat #11304-011). The 50 μL reaction mixture consisted of 40 ng of genomic P. falciparum NF54 template, 0.2 mM dNTP mix, 2.0 mM MgSO₄, 1 μM of each primer and 1 U Taq polymerase in 1× reaction buffer. Following initial denaturation at 94° C. for 90 seconds, PCR was performed with 35 cycles of 15 seconds at 94° C., 35 seconds at 56° C., and 5 minutes at 68° C.

The resulting 706 base pair phosphates sequence was sub-cloned into the pGEM-T Easy® vector (Promega) using the manufacturer's protocol; 5 μL Rapid ligation buffer, 1 μL pGEM-T Easy® cloning vector, 3 μL of the PCR product and 1 μL DNA ligase. The reaction was performed at 4° C. overnight and then 5 μL was used to transform 25 μL of XL-10 Gold chemically competent Escherichia coli.

After the addition of the ligation reaction to the E. coli, the bacteria were then incubated on ice for 30 minutes. Then heat-shocked for 30 seconds at 45° C. before chilling on ice for 2 minutes. Then 250 μL of S.O.C. medium (Invitrogen, cat#15544-034) was added to the bacteria before shaking at 200 rpm for 1 hour at 37° C. The bacteria were spread on warmed LB agar plates with 50 μg/mL ampicillin.

Bacterial clones were screened by DNA extraction and restriction digest. Each bacterial clone was used to inoculate 5 mL of LB with 50 μg/mL ampicillin and grown to saturation. A sample of each bacterial clone was patched to a fresh LB agar plate and kept till after screening. After incubation 1-1.5 mL of the bacterial culture was transferred to a microcentrifuge tube and centrifuged for 5 minutes at 6000 rpm to pellet the bacteria. After aspirating the supernatant, 100 1 μL of lysis buffer (10 mM Tris-HCl pH 8.0, 1 mM EDTA, 15% sucrose w/v, 2 mg/mL lysozyme, 0.2 mg/mL pancreatic RNAse and 0.1 mg/mL BSA) was added to each sample and incubated at room temperature for 5 minutes. Following incubation the samples were placed in a boiling water bath for 60 seconds, then on ice for another 60 seconds. The bacterial debris was spun down at 15000 rpm for 15 minutes and the supernatant containing the DNA was collected for analysis.

The DNA samples were screened by a restriction digest method using BamHI and EcoRI. Each 20 μL digest reaction contained 10 μL of the DNA, 2 μL of the 10× reaction buffer, 100 U of each restriction enzyme and the appropriate volume of water to bring it up to the final volume. The reaction was incubated at 37° C. for 1 hour and then analyzed by agarose (0.8%) gel electrophoresis. The clones that showed the correct insert were then grown up in a LB broth culture and the DNA was extracted using the Wizard® Plus SV Minipreps Kit (Promega, cat# A1460) using the manufacturer protocol. The purified plasmid DNA samples were then sequenced for verification.

The verified pure plasmid DNA samples were restriction digested with BamHI and EcoRI then gel extracted before ligation to pGEX-2T (GE Healthcare) using 2 μL of the vector (pGEX-2T), 6 μL of the entry sequence, 2 μL 10× ligation buffer, 1 μL ATP, and 400 U T4 ligase. The reaction was incubated overnight at 16° C. Following the ligation reaction, 10 μL was used to transform 25 μL of chemically competent XL-10 Gold E. coli. The resulting clones were screened by restriction digest as previously and then sequenced for verification. The pGEX-2T plasmid with the correct insert was then used to transform BL21(DE3)pLysE chemically competent E. Coli, by the same method used previously.

Expression and Purification of the PF13_{—}0027 Phosphatase Domain Using BL21(DE3)pLysE E. Coli

A frozen stock of the appropriate bacterial clone containing the phosphatase recombinant plasmid was used to transform a starter culture of LB, which was incubated overnight to saturation. The starter culture was diluted 1:20 in fresh Terrific Broth (TB; 12 g tryptone, 24 g yeast extract, 4 mL glycerol, 0.17M KH2PO4 and 0.72M K2HPO4 in 1 L distilled water). The newly diluted bacterial culture was grown up to mid-log phase (OD₆₀₀=0.4-0.5). Expression was then induced with 1 mM Isopropyl β-D-1-thiogalactopyranoside (IPTG). Then grown for an additional 5 hours. Following expression the bacterial were pelleted by centrifugation at 6000 rpm for 15 minutes at 4° C.

The bacterial pellets were resuspended in 5 mL/g bacterial pellet of chilled lysis buffer (1×PBS with 1 mM PMSF, 1 mg/mL lysozyme, 1:1000 protease inhibitor cocktail Sigma #P8849-5ML). The suspension was incubated on ice for 15 minutes (bacterial lysis) then sonicated 3 times for 30 seconds. Fifteen μg/mL DNAse and 2 mM MgCl2 with 1% Triton X-100 (final concentration) was added. The suspension was incubated for another 15 minutes (digest DNA) and sonicated 3 times for 30 seconds each. The bacterial debris was removed by centrifugation for 30 minutes at 20000×g and the remaining supernatant with the soluble recombinant protein was collected. A 50% glutathione bead (Invitrogen, cat#G2879) slurry was prepared and 500 μL was added to the supernatant. Binding was carried out at room temperature for 1 hour with shaking. Following binding, the beads were pelleted by centrifugation at 1800 rpm and washed three times with 10 bed volumes of PBS. The bound GST fusion recombinant protein was eluted with 50 mM reduced L-Glutathione in PBS pH 8.5, and incubated for 1 hour with shaking at 37° C.

When removing the GST fusion using thrombin, the agarose beads were suspended in 1 mL of 1×PBS with 50 U of thrombin protease and incubated at room temperature overnight while shaking. Following overnight cleavage the beads were pelleted by centrifugation at 1800 rpm before removing the supernatant with the cleaved phosphatase domain.

Protein samples were analyzed by SDS-PAGE and Western blotting to identify the 52 kDa recombinant GST fusion phosphatase or the 28 kDa cleaved phosphatase domain. As a negative control, a non-induced bacterial sample was prepared and analyzed along with the affinity-purified samples. In order to prepare each sample for electrophoresis, a 1:1 mixture of protein to protein loading dye was mixed and warmed for 5 minutes at 80° C. and 10 μL was loaded onto the gel. For Western blot, 5 μL was loaded on the gel. Protein gels were stained with coomassie brilliant blue for 30 minutes then destained for two hours with destain buffer.

Mass Spectrometry Analysis of the Recombinant PF13_{—}0027 Phosphatase Domain

Following SDS-PAGE, the gel was washed 2 times for 10 minutes with distilled water. Each band to be identified was excised along with a negative control from a region of the gel without any protein. Following the initial wash and excision of the gel bands, further washing was carried out to remove SDS. Each gel slice was then washed with 200 μL of 50% acetonitrile (ACN) in distilled water while vortexing for 15 minutes. The wash step above was repeated a second time. After the second wash, each gel slice was washed with 100% ACN for 10 minutes. The gel pieces were rehydrated with 50 μL of 100 mM ammonium bicarbonate (ABC) for 5 minutes. An equal volume of CAN was subsequently added to get an equal ratio of ACN/ABC and votexed for 15 minutes. The wash was removed using a speedvac for 15 minutes.

Each protein sample was reduced and alkylated in the gel. Each gel piece was rehydrated with 100 uL of 45 mM DTT at 55 C for 30 minutes. After incubation, the buffer was discarded and the tubes were chilled to room temperature and covered with fresh iodoacetaminde. The gel slices were incubated in the dark for 30 minutes at room temperature. The gel pieces were washed 3 times with 100 uL 50% ACN/50 mM ABC with agitation for 15 minutes until the gel pieces were colorless. Each gel piece was dried with the speedvac for 15 minutes followed by trypsin digest.

The trypsin digest buffer was prepared with 12 ng/uL of Promega trypsin in 50 mM ABC. Each gel piece was covered with the trypsin digest buffer and incubated on ice for 45 minutes. The gel pieces were moved to 37 degrees and incubated overnight. After overnight incubation the reaction was stopped using 5% glacial acetic acid (final concentration). One hundred uL of 50:50 ACN:water containing 1% formic acid was used to cover the gel pieces. Each sample was sonicated for 15 minutes, and the supernatant was transferred to a new clean tube. Using the speedvac, the sample was dried for 15 minutes until the tube was completely dry. The samples were resuspended in 0.1% formic acid to get a final protein concentration of approximately 1 μmol/mL (20-40 uL). The samples were transferred to clean vials for analysis by mass spectrometry.

Following Orbitrap MS the data was loaded into Mascot (Matrix Science) and the protein fragments were compared to the proteomic database and analyzed further using Scaffold (Proteome Software Inc.) protein identification software.

PF13_{—}0027 Antisera Production

Affinity purified samples were sent to Cocalico BIologicals Inc. for antisera production. The antisera was produced using rat specimens that were prescreened to determine the specimen with minimal background reaction. The selected rat was pre-bled at day 0 and inoculated with Titermax. On days 14 and 21 the rat was given a Titermax boost and a test bleed was drawn at day 35. The test bleed was analyzed by western blot and IFA against the wild type, mutant and complemented mutant parasite protein extracts. The rat was given a final Titermax boost at day 49 and then another test bleed was taken at day 56. The test bleed was analyzed as previously. Exsanguination was carried out on day 60 for the extraction of the final sera sample.

IFA (Immunofluorescent Assay) for P. falciparum NF54

Parasite cultures of NF54 schizonts were harvested and resuspended in PBS and fetal bovine serum. Two μL of each parasite pellet was smeared on a glass slide and fixed for 5 min at room temperature with a 9:1 Acetone:methanol solution. Once dry the slides were incubated for 1 hour with 500 μL of 1% triton X-100 in PBS. Following incubation the slides were washed 5 times for 5 minutes with 500 μL PBS. The slides were then incubated in 3% BSA for 1 hour at room temperature. The slides were washed 2 times for 5 min with PBS. A 1:100 dilution of the primary antibody (custom rat anti-PF13_{—}0027, Cocalico Biologicals Inc.) was prepared and 500 1 μL was added to the slides. The slides were incubated with the primary antibody for 1 hour at room temperature followed by five 5-minute washes with PBS. The secondary antibody (FITC-goat-anti-rat IgG, Invitrogen Cat#62-9511) was prepared by diluting it 1:70 in a solution of PBS and normal goat serum (0.60 mg/mL final) and incubating for 1 hour at room temperature in the dark. After incubation the slides were washed 5 times for 5 minutes each with PBS in the dark. The slides were mounted using 100 μL Fluormount G (Southern Biotech cat #0100-01) and examined using the DeltaVlsion Core fluorescent microscope.

Immunoprecipitation of PF13_{—}0027

To extract the parasite protein extracts for analysis, 60 mL cultures were grown and centrifuted to harvest the parasitized RBCs. The parasite cultures were saponin-treated and washed with PBS. The parasites were solubilized in RIPA (1% Triton X-100, 1% sodium deoxycholate, 0.1% SDS, 150 mM NaCl, 25 mM Tris-HCl, pH 8, in phosphate-buffered saline). The parasite protein extracts were analyzed by Western blotting and IFA. Binding of RIPA supernatant to goatpolyclonal HA Ab-coated agarose beads was performed overnight. The protein extracts were washed three times with 0.5% NP-40, 50 mM Tris (pH 7.4), 150 mM NaCl, 5 mM EDTA and eluted directly by boiling for 3 min in SDS-PAGE sample buffer.

Transposon Mutagenesis Created a P. falciparum Mutant with Attenuated Blood-Stage Growth

Malaria parasites developmental stages have rigid patterns of gene expression with few obvious regulatory mechanisms. The P13_{—}0027 PTP product has a major role in the regulation of the P. falciparum blood stages and all developmental stages at the critical transition from trophozoite to the schizont phase, which is equivalent to G1 to S/G2 transition, thereby extending the cell cycle in the PTP null parasite by >10% and causing serious developmental defects. These characteristics make the function of P13_{—}0027 somewhat similar to CDC25 with a rhodanese domain in tandem with the dual specificity phosphotyrosine phosphatase domain. Disruption of the normal progression of development leads to two significant phenotypes, which renders the KO clone able to grow at only 30% of wild type NF54. First, early lysis of the parasitophorous vacuole membrane releases the developing schizont into the erythrocyte cytoplasm. Second, and more importantly, after schizont rupture, most merozoites remain non-invasive and unable to complete or even initiate the invasion process.

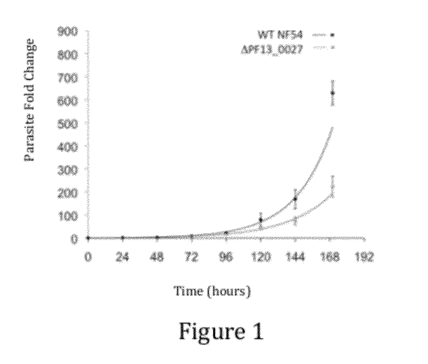

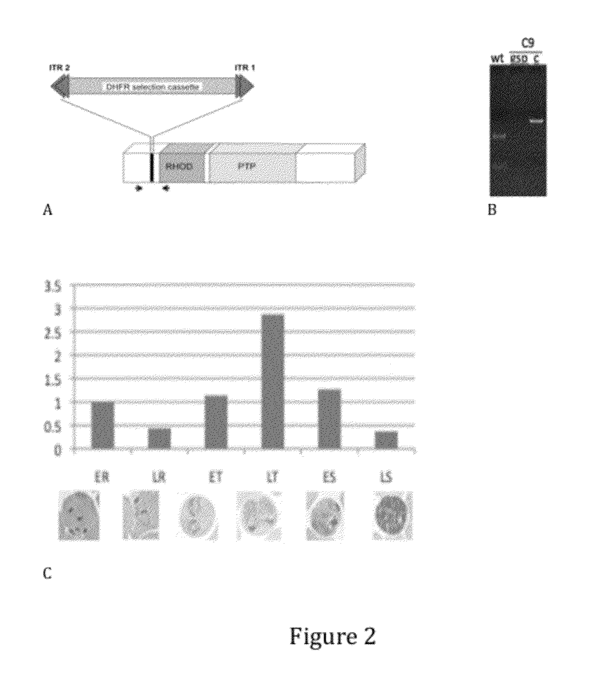

The laboratory line of P. falciparum NF54 was subjected to random insertion mutagenesis using the transposon piggyBac carrying a hdhfr drug resistance cassette. Clones of mutant parasites were isolated by drug selection after limiting dilution and screened for slow growth. This preliminary screen identified mutant clone C9 that has significantly attenuated blood-stage growth. The mean doubling time of the C9 mutant was much longer (≈21 hours) than the parent NF54 (≈18.4 hours) resulting in net growth rate in C9 decreased by >60% as shown in FIG. 1. This stable attenuated growth phenotype resulted from a single transposon integrated at a TTAA within the open reading frame of the PF13_{—}0027 locus that effectively knocked out expression of this gene as determined by RT-PCR (FIG. 2a, 2b). The PF13_{—}0027 is a unique conserved gene present as a single copy in all Plasmodium genomes sequenced to date. Transcript abundance in P. falciparum NF54 blood-stage development peaked in late trophozoite stages (FIG. 2c) in a pattern similar to that observed in P. vivax blood stages.

Structure Characteristic of a CDC25-Like Dual Specificity Phosphatase

Analysis of the single open reading frame of PF13_{—}0027 identified an N-terminal rhodanese (RHOD) domain followed in tandem by a dual-specificity protein tyrosine phosphatase II (PTP) domain. A previous analysis of the P. falciparum phosphatome identified PF13_{—}0027 rhodanese domain (SEQ ID NO:3) as closely related to human CDC25s, except the malarial protein lacked a critical residue needed for catalytic activity. The rhodanese domain is inactive due to the substitution of the catalytic cysteine at position 144 with an aspartic acid. Based upon the consensus structure of a PTP (Pyst1) the catalytic residues of dual specificity phosphatase domain (SEQ ID NO:4) are Aspartic acid, Cysteine, and Arginine. PF13_{—}0027 (SEQ ID NO:1) possesses the first two of the three of these residues at positions 345 and 382, respectively, but Arginine is replaced by Isoleucine at position 398. These characteristics partially support the classification of this protein as a dual specificity phosphotyrosine phosphatase, possibly in the CDC25 superfamily, although presence of an inactive rhodanese domain is also consistent with dual specificity MAPK phosphatases. An insertion of amino acids unique to the Plasmodium PTP between the residues 383 and 397 identify the PF13_{—}0027 product as distinct from its orthologs in other species.

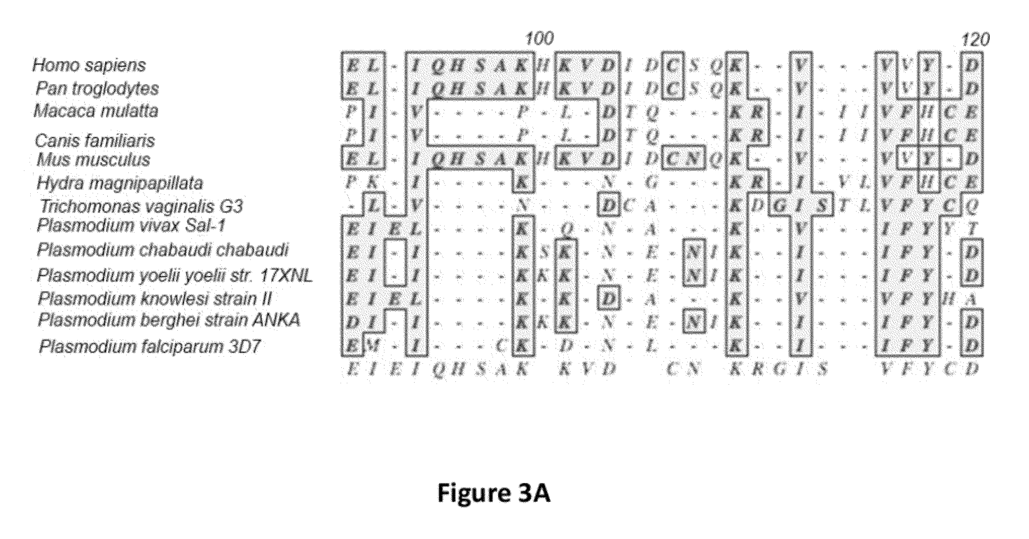

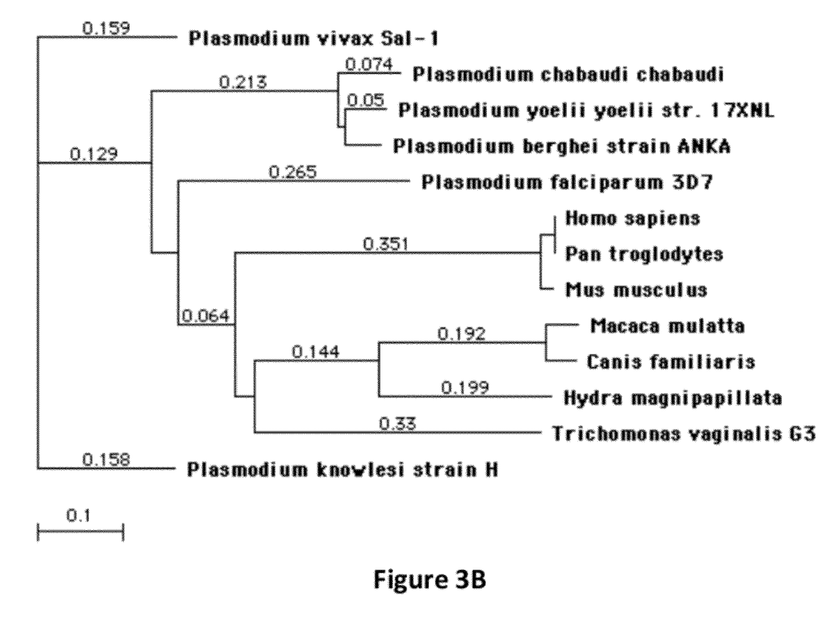

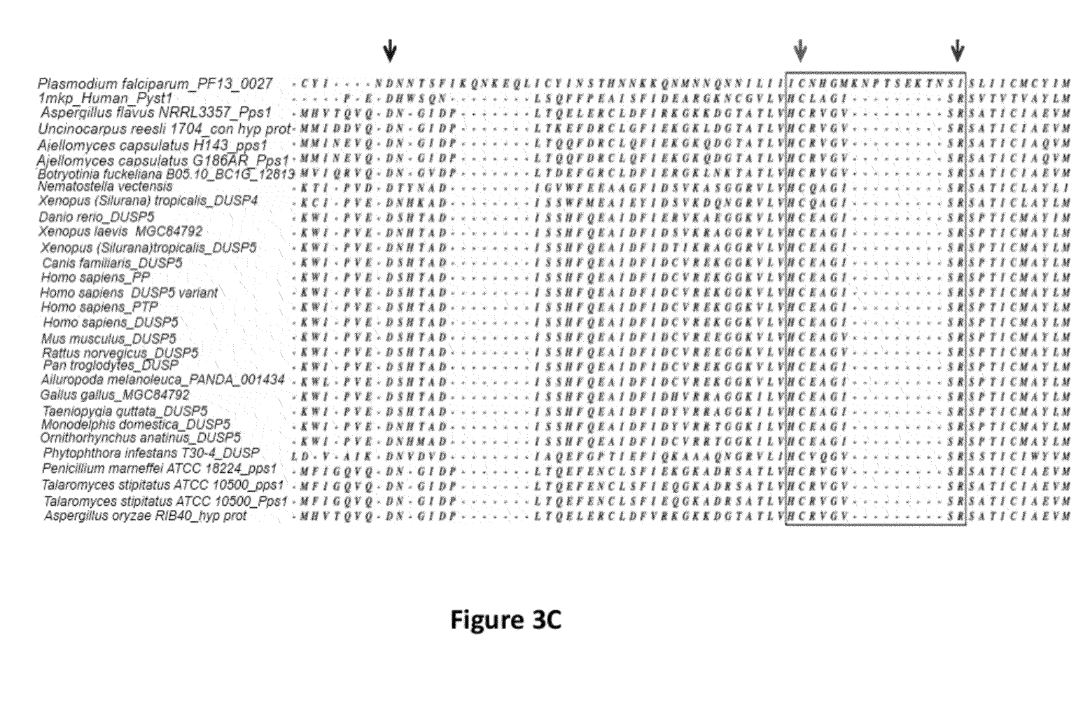

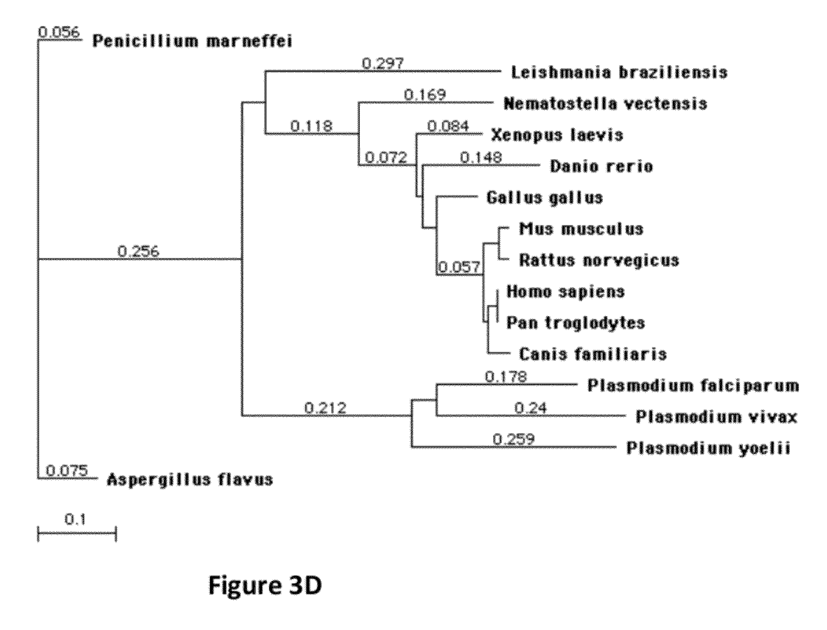

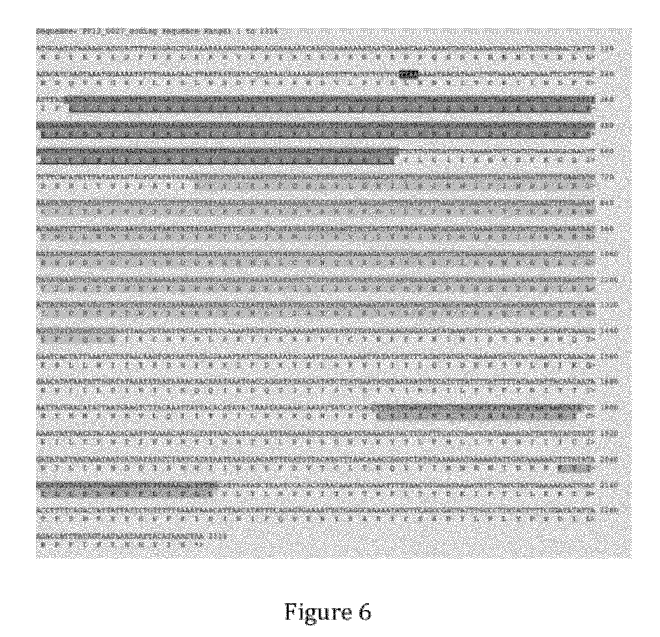

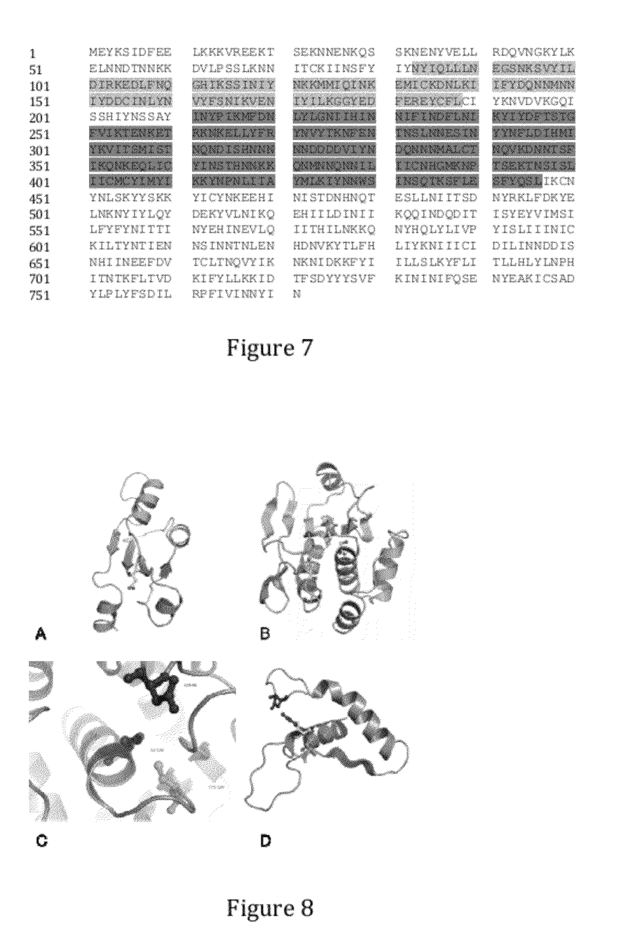

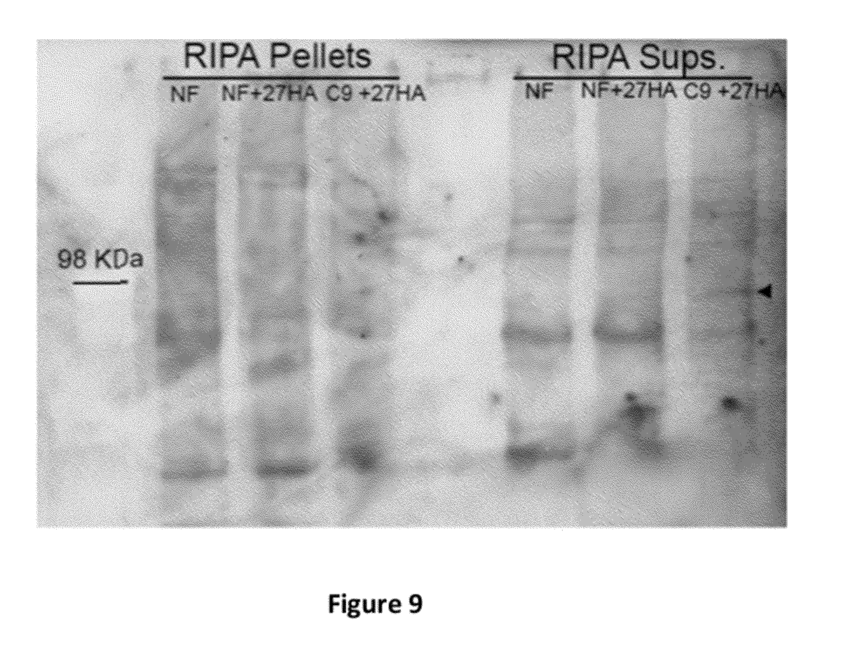

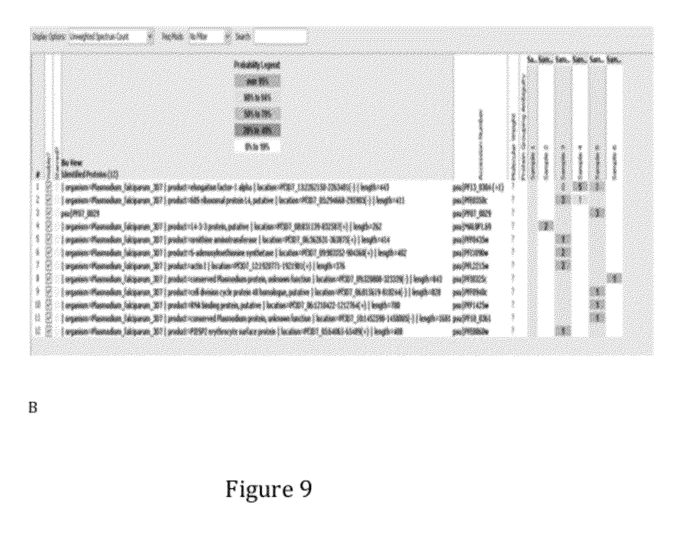

The presence of the inactive rhodanese domain is consistent with dual specificity MAPK phosphatases. While catalytic RHOD are characteristic of CD25 phosphatases that regulate cell cycle progression, non catalytic RHOD usually have a regulatory role. CDC25 is also regulated by some dual specificity MAPK phosphatases in higher eukaryotes that have a similar tandem arrangement of a RHOD and PTP domain. FIG. 3A (SEQ ID NOs: 3 and 7-19) as well as the tree shown in FIG. 3B, depict the sequence alignment of the rhodanese domain of P13_{—}0027 with other domains from various species. It was found that the sequence alignment of the deduced PF13_{—}0027 PTP sequence (SEQ ID NO:4) is relatively weak with other PTP and its relatedness is distant (FIG. 3C (SEQ ID NOs: 4 and 20-34); FIG. 3D). Most importantly the distinctive signature motif, HCxxGxxR, that is characteristic of the catalytic region of dual specificity phosphatases is not fully conserved (FIG. 3). In the deduced primary sequence of PF13_{—}0027 the conserved C383 (arrow) aligns with the other phosphatases, but the Histidine of the signature motif is replaced with I382 (arrow) and the conserved Serine (arrow) is shifted out of alignment. A homology model with the closest structural neighbor, MKP1 of humans (FIG. 9) suggests that D345/C383/5399 match the structural positions of the signature PTP motif (FIGS. 6, 7, 8).

PF13_{—}0027 is Critical for Cell Progression into S Phase

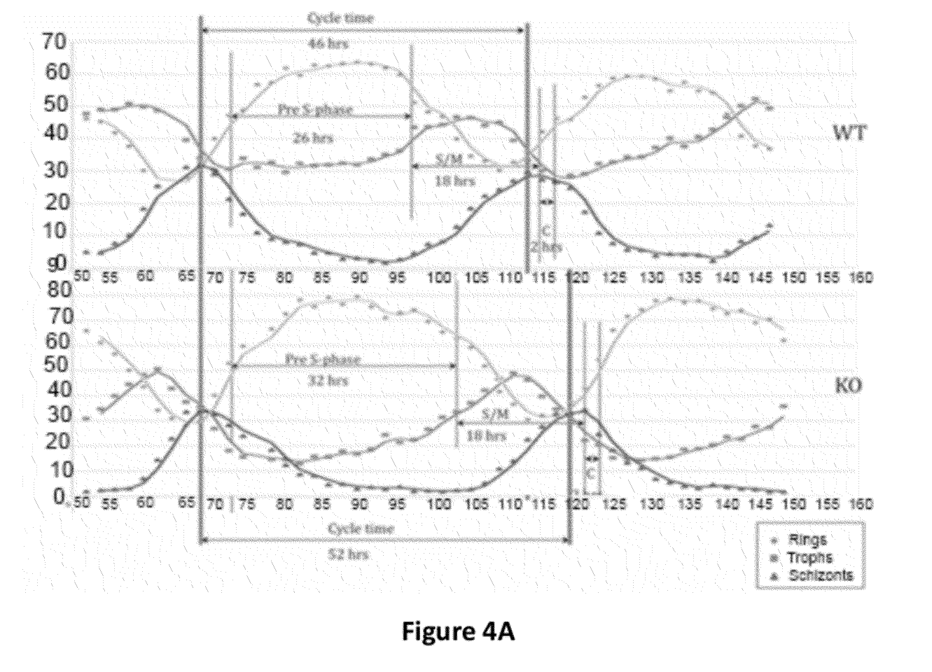

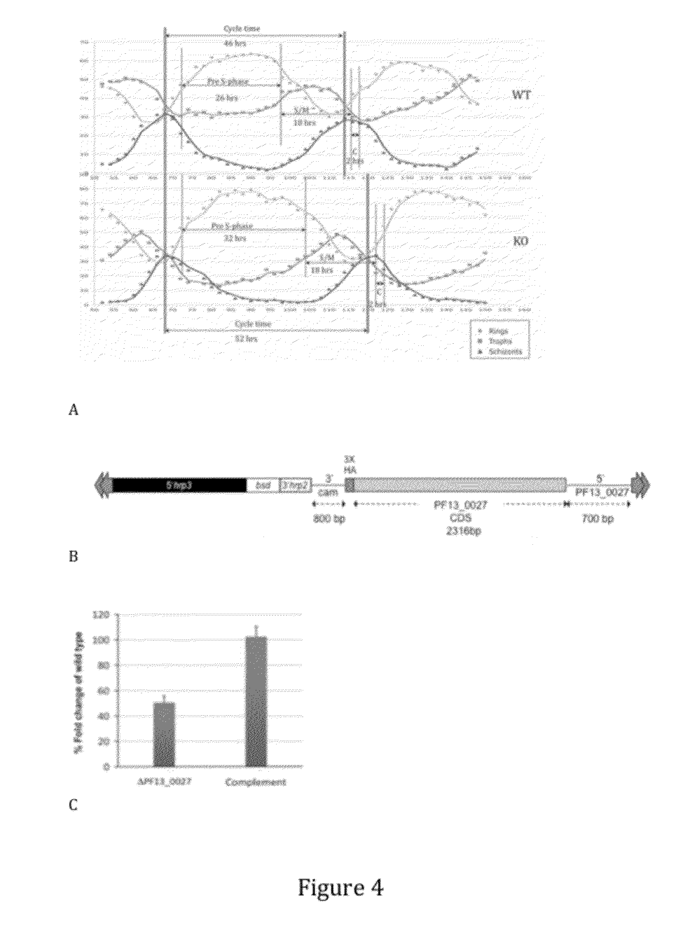

To help elucidate a function, a more detailed growth analysis of the C9 mutant was performed to determine if the null phenotype alters the normal cell cycle of blood-stage P. falciparum. Surprisingly, the time to complete asexual development of the null mutant was significantly longer (52 hrs) compared to its wild-type parent (46 hrs). The extended cycle time in the null mutant malaria parasites was due entirely to an extended 32 hrs pre-S phase (G1), or trophozoite stage, versus 26 hrs for wild-type parasites (FIG. 4). The final phases of the development cycle, early/late schizont or S/G2/M phases, were not different in the mutant and wild-type parasites.

Phenotype Rescue by Genetic Complementation

The observed phenotype was due to the disruption of PF13_{—}0027 as shown by the results of the process where a full-length copy of the wild type gene was inserted the genome of the C9 mutant using a piggyBac element. The intact PF13_{—}0027 transgene restored normal growth time and growth pattern thus rescuing the wild-type phenotype, validating that this knockout severely attenuates the asexual developmental cycle.

Complex Phenotype Alters Normal Egress And Invasion

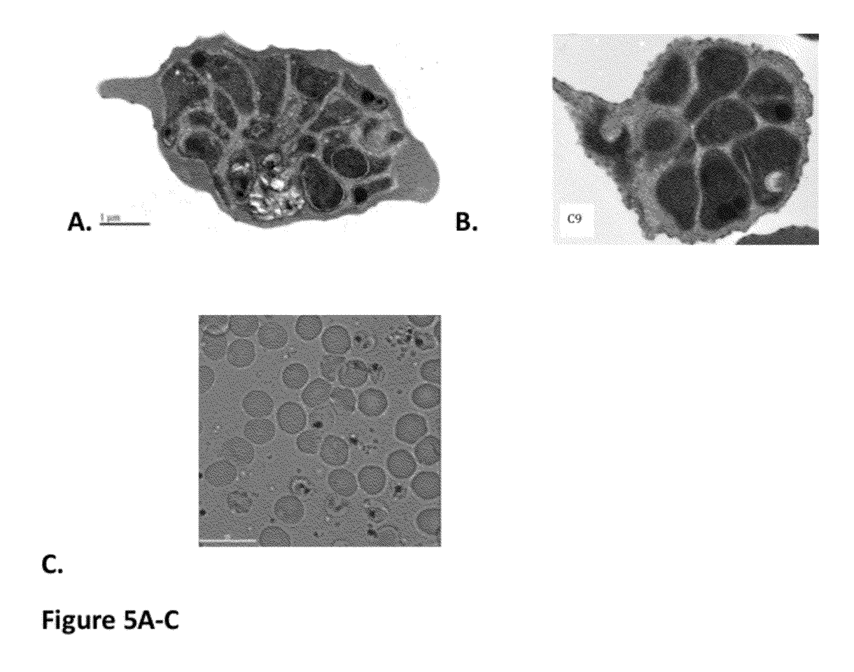

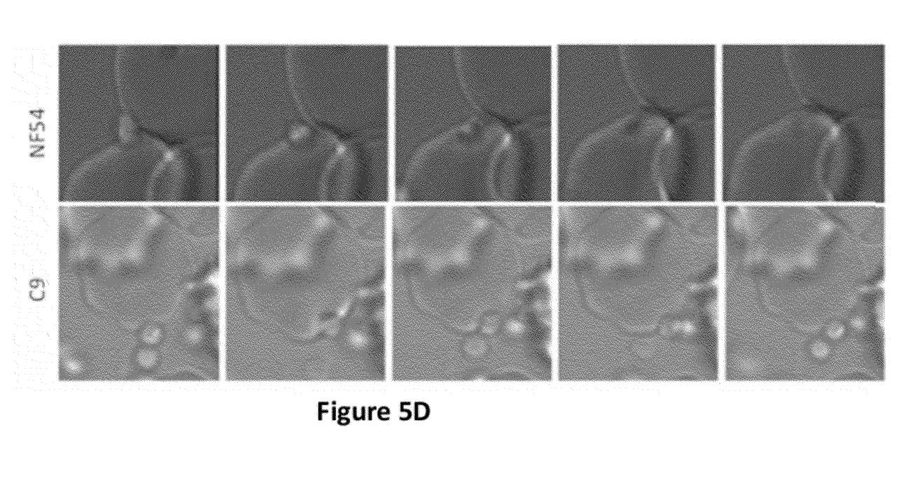

Since alteration of a phosphorylation pathway can have significant downstream consequences the inventors analyzed the C9 for other changes in the wild-type development pattern. Morphology of parasite development was similar to wild-type parasites but the cascade of metabolic processes disrupted by the knockout of PF13_{—}0027 adversely changed the final stages of development (egress) and severely compromised the ability of most merozoites to invade new erythrocytes. Normal egress of P. falciparum explosively releases intracellular merozoites by simultaneous disruption of the internal parasitophorous vacuole membrane (PVM) and the erythrocyte limiting membrane. Ultrastructural analysis revealed the PVM was prematurely degraded in many of the schizont nearing final segmentation leaving merozoites free in the erythrocyte cytoplasm (FIG. 5). Premature loss of the PVM may adversely affect the post-translation modifications of merozoite surface proteins. Unusually large numbers of merozoites accumulated in the culture supernatants of the mutant C9 often remaining in loose association at the site of the ruptured schizont (FIG. 5c). Merozoite invasion of erythrocytes is typically a rapid process (<30 s) that initiated by contact, merozoite apical reorientation to erythrocyte surface, junction formation and entry of the parasite into the erythrocyte via a moving junction. All observed invasion aborted midway through this process after the parasite indented the erythrocyte surface (FIG. 5d). These late-stage phenotypes were not observed after PF13_{—}0027 function was restored with the genetic rescue.

Protein Interactions

Protein-protein interactions help define processes and pathways through which proteins function. Protein interactions of the PF13_{—}0027 product were investigated using 3HA-tagged transgene product to rescue the mutant phenotype. Expression of the transgene was confirmed by western blot by an anti-HA antibody (FIG. 9a). Proteins isolated by pull downs with the HA-tag were separated by SDS-PAGE and unique bands identified by mass spectrometry (FIG. 9b). Multiple peptide fragments were identified for elongation factor 1a and protein 14-3-3, which are typically associated with CDC25 and regulate its function related to initiating the G1-S transition.

Genetic Validation Experiment

The positions of critical residues as determined by in silico docking analysis and the results of wet lab in vitro assays provides a basis for target site mutagenesis experiments to characterize and define the PTP active site of PF13_{—}0027. The docking process begins with CDC25 phosphatase inhibitors, examining all the possible stereoisomers and orientations of the ligand binding site based on the pocket identified in 1 mkp from the PDB. Genetic validation of the PTP phosphatase domain (rPTP) active site helps optimize lead compounds that target the active site residues least capable of undergoing changes that may lead to future resistance.

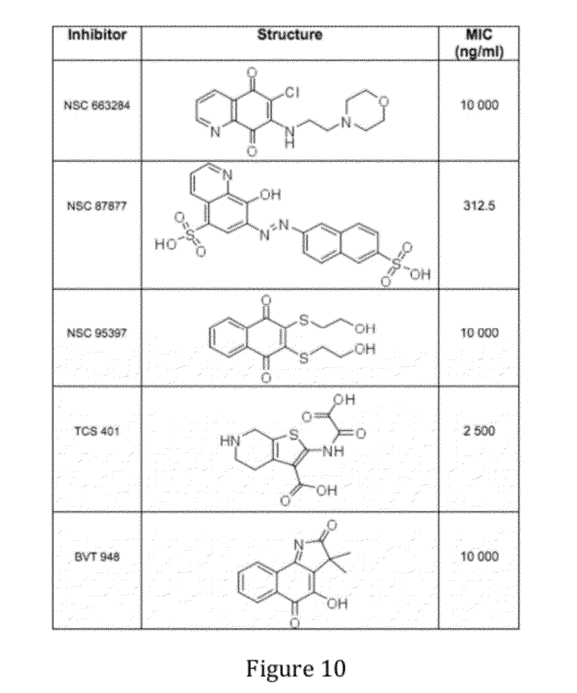

Minimum inhibitory concentration (MIC) assays were carried out in vitro to get a baseline level of resistance for the drugs (FIG. 10). Current phosphatase inhibitors were used to test their effects on the growth of P. falciparum. These phosphatase inhibitors are known to act against CDC25s and dual specificity protein tyrosine phsophatases such as shp1 and shp2.

The design of inhibitors depends on molecular modeling-guided experimentation to further optimize the biological activity and especially the selectivity of these novel phosphatase inhibitors. The GLIDE program (Schrödinger, LLC) is employed to serve as the foundation for docking studies performed using homology models of PF13_{—}0027 PTP constructed using PRIME (Schrödinger, LLC). GLIDE is well suited for the investigations since studies comparing various docking methods rank GLIDE among the most accurate. Nonetheless, other docking software (e.g., AutoDock, FlexX and GOLD) can be utilized for consensus scoring. A variant of GLIDE known as CombiGLIDE is used to aid in the design of chemical libraries for lead optimization. CombiGLIDE allows the user to define a docked scaffold as a template upon which user-defined substituents are added combinatorially to user-defined attachment sites to generate a library of structures that are then docked to the protein using GLIDE.

Structure-property relationship (SPR) data is integrated in the iterative process of inhibitor design and optimization. Especially, solubility and permeability is routinely determined experimentally for each synthesized compound to obtain solid SPR data. Simple LC/MSbased SPR assays have been implemented in the Manetsch laboratory and a third-party for additional or in depth SPR data is also used. In order to produce compounds that are likely to have optimal ADME characteristics, the QikProp program (Schrödinger, LLC) is employed. QikProp is based upon linear correlations previously established between a number of ADME properties and both 2D and 3-D descriptors calculated for a “training set” of known drugs with experimentally determined ADME properties (ca. 700 compounds). The calculation of relevant 2-D and 3-D descriptors for the compound of interest, provides in silico prediction of the experimental ADME properties for the molecule that includes Caco-2 cell permeability, aqueous solubility, log Poctanol/water, and human serum albumin binding.

Initial screening for lead candidates uses a microplate assay using recombinant PTP of PF13_{—}0027 and the identified orthologue of P. vivax (PVX_{—}122110). As a counter screen the human PTP domain of 1 mkP that served as the template for the initial homology model was used. Synthetic codon optimized genes encoding the PTP ORFs are cloned into pET-21a(+) and transformed in E. coli BL21(DE3)ₚLysE. Diluted cultures are induced with IPTG, protein extracted from the cell pellet is purified using HisTrap HP columns on an AKTA Explorer 10, eluted using a linear gradient of imidazole, desalted and rPTPs concentrated using Amicon filter devices. Standard methods for refolding are used if needed. A commercial colorimetric assay is used to screen for phosphatase inhibitory activity in a microplate assay.

Manual methods are initially used and depending on throughput needed, robots were available for large-scale screening. Putative inhibitors identified via microplate assay screening are confirmed in standard cell based in vitro growth inhibition assays with multidrug resistant P. falciparum. In vivo drug screening was available using rodent malaria models for erythrocytic and exoerythrocytic stage (P. berghei, P. y. yoelii). An insectary suite is available for production of P. falciparum sporozoites that can be utilized for transmission blocking and liver studies. FIG. 10 illustrates some known CDC25 and PTP inhibitors that may be used to target P13_{—}0027.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between. Now that the invention has been described,

What is claimed is:

1. A method of treating malaria comprising contacting a cell infected with a Plasmodium species with a therapeutically effective amount of a phosphatase inhibitor.

2. The method of claim 1 wherein the cell is selected from the group consisting of erythrocytes and hepatocytes.

3. The method of claim 1 wherein the phosphatase inhibitor is selected from the group consisting of CDC25 phosphatase inhibitors and dual specificity protein tyrosine phosphatase inhibitors.

4. The method of claim 1 wherein the phosphatase inhibitor targets a protein gene in a Plasmodium species wherein the protein has an amino acid sequence having homology to SEQ ID NO: 1.

5. The method of claim 1 wherein the Plasmodium species is selected from the group consisting of P. falciparum and P. vivax.

6. A method of preventing malaria comprising regulating the cell cycle of a Plasmodium species by inhibiting the expression of the P13_{—}0027 protein.

7. The method of claim 6 wherein the Plasmodium species is selected from the group consisting of P. falciparum and P. vivax.

8. The method of claim 6 wherein the P13_{—}0027 protein has an amino acid sequence having homology to SEQ ID NO: 1.

9. The method of claim 6 wherein the expression of the P13_{—}0027 protein is inhibited by a phosphatase inhibitor.

10. The method of claim 9 wherein the phosphatase inhibitor is selected from the group consisting of CDC25 phosphatase inhibitors and dual specificity protein tyrosine phosphatase inhibitors.

11. A method of preventing malaria comprising regulating the cell cycle of a Plasmodium species by inhibiting the expression of the P13_{—}0027 gene.

12. The method of claim 11 wherein the Plasmodium species is selected from the group consisting of P. falciparum and P. vivax

13. The method of claim 11 wherein the expression of P13_{—}0027 is inhibited by the insertion of a genetic element in the open reading frame.

14. The method of claim 13 wherein the genetic element is selected from the group consisting of nucleic acids and transposon sequences.

15. The method of claim 13 wherein the genetic element is inserted into a TTAA sequence in the open reading frame.

16. A method of preventing malaria comprising administering a therapeutically effective amount of a PTP-null Plasmodium species and a pharmaceutically acceptable carrier.

17. The method of claim 16 wherein the PTP-null Plasmodium species has the insertion of a genetic element in the open reading frame.

18. The method of claim 17 wherein the genetic element is selected from the group consisting of nucleic acids and transposon sequences.

19. The method of claim 17 wherein the genetic element is inserted into a TTAA sequence in the open reading frame.

20. A pharmaceutical composition for preventing malaria comprising a PTP-null Plasmodium species and a pharmaceutically acceptable carrier.

21. The composition of claim 20 wherein the PTP-null Plasmodium species has the insertion of a genetic element in the open reading frame.

22. The composition of claim 21 wherein the genetic element is selected from the group consisting of nucleic acids and transposon sequences.

23. The composition of claim 21 wherein the genetic element is inserted at a TTAA sequence in the open reading frame.


SEQUENCE LISTING (36 sequences)

SEQ ID NO: 1 - protein, 771 aa, artificial sequence
  Protein for P. falciparum knockout
MEYKSIDFEELKKKVREEKTSEKNNENKQSSKNENYVELLRDQVNGKYLKELNNDTNNKKDVLPSSLKNNITCKIINSFYIYNYIQLLLNEGSNKSVYILDIRKEDLFNQGHIKSSINIYNKKMMIQINKEMICKDNLKIIFYDQNNMNNIYDDCINLYNVYFSNIKVENIYILKGGYEDFEREYCFLCIYKNVDVKGQISSHIYNSSAYINYPIKMFDNLYLGNIIHINNIFINDFLNIKYIYDFTSTGFVIKTENKETRKNKELLYFRYNVYTKNFENTNSLNNESINYYNFLDIHMIYKVITSMISTNQNDISHNNNNNDDDDVIYNDQNNNMALCTNQVKDNNTSFIKQNKEQLICYINSTHNNKKQNMNNQNNILIICNHGMKNPTSEKTNSISLIICMCYIMYIKKYNPNLIIAYMLKIYNNWSINSQTKSFLESFYQSLIKCNYNLSKYYSKKYICYNKEEHINISTDNHNQTESLLNIITSDNYRKLFDKYELNKNYIYLQYDEKYVLNIKQEHIILDINIIKQQINDQDITISYEYVIMSILFYFYNITTINYEHINEVLQIITHILNKKQNYHQLYLIVPYISLIIINICKILTYNTIENNSINNTNLENHDNVKYTLFHLIYKNIIICIDILINNDDISNHIINEEFDVTCLTNQVYIKNKNIDKKFYIILLSLKYFLITLLHLYLNPHITNTKFLTVDKIFYLLKKIDTFSDYYYSVFKININIFQSENYEAKICSADYLPLYFSDILRPFIVINNYIN

SEQ ID NO: 2 - DNA, 2316 bp, artificial sequence
  Knockout DNA sequence for P13_0027 gene
atggaatataaaagcatcgattttgaggagctgaaaaaaaaagtaagagaggaaaaaacaagcgaaaaaaataatgaaaacaaacaaagtagcaaaaatgaaaattatgtagaactattgagagatcaagtaaatggaaaatatttgaaagaacttaataatgatactaataacaaaaaggatgttttaccctcctccttaaaaaataacataacctgtaaaataataaattcattttatatttataattacatacaactattattaaatgaaggaagtaacaaaagtgtatacatattagatattcgaaaagaagatttatttaaccagggtcatattaagagtagtattaatatatataataaaaagatgatgatacaaataaataaagaaatgatatgtaaagacaatttaaaaattatcttttatgatcagaataatatgaataatatatatgatgattgtattaatttatataatgtctatttttcaaatattaaagtagagaacatatacattttaaaaggaggatatgaagattttgaaagagaatattgtttcttgtgtatttataaaaatgttgatgtaaaaggacaaatttcttcacatatttataatagtagtgcatatataaattatcctataaaaatgtttgataacttatatttaggaaacattattcatataaataatatttttataaatgattttttgaacatcaaatatatttatgattttacatcaactggttttgttataaaaacagaaaataaagaaacaaggaaaaataaggaacttttatattttagatataatgtatatactaaaaattttgaaaatacaaattctttgaataatgaatctattaattattacaattttttagatatacatatgatatataaagttattacttctatgataagtacaaatcaaaatgatatatctcataataataataataatgatgatgatgatgtaatatataatgatcagaataataatatggctttatgtacaaaccaagtaaaagataataatacatcatttataaaacaaaataaagaacagttaatatgttatataaattctacacataataacaaaaaacaaaatatgaataatcaaaataatatccttattatatgtaatcatggaatgaaaaatcctacatcagaaaaaacaaatagtataagtcttattatatgtatgtgttatattatgtatataaaaaaatataaccctaatttaattattgcctatatgctaaaaatatataataactggagtataaattctcagacaaaatcatttttagaaagtttctatcaatccctaattaagtgtaattataatttatcaaaatattattcaaaaaaatatatatgttataataaagaggaacatataaatatttcaacagataatcataatcaaacggaatcactattaaatattataacaagtgataattataggaaattatttgataaatacgaattaaataaaaattatatatatttacagtatgatgaaaaatatgtactaaatatcaaacaagaacatataatattagatataaatataataaaacaacaaataaatgaccaggatataacaatatcttatgaatatgtaataatgtccatcttattttatttttataatattacaacaataaattatgaacatattaatgaagtcttacaaattattacacatatactaaataagaaacaaaattatcatcagctttatttaatagttccttacatatcattaatcataataaatatatgtaaaatattaacatacaacacaattgaaaacaatagtattaacaatacaaatttagaaaatcatgacaatgtaaaatatactttatttcatctaatatataaaaatattattatatgtattgatatattaataaataatgatgatatatctaatcatataattaatgaagaatttgatgttacatgtttaacaaaccaggtctatataaaaaataaaaatattgataaaaaattttatataatattattatcattaaaatattttcttataacacttttgcatttatatcttaatccacacataacaaatacgaaatttttaactgtagataaaatattctatctattgaaaaaaattgataccttttcagactattattattctgtttttaaaataaacattaacatatttcagagtgaaaattatgaggcaaaaatatgttcagccgattatttgcccttatatttttcggatatattaagaccatttatagtaataaataattacataaactaa

SEQ ID NO: 3 - protein, 24 aa, artificial sequence
  Amino acid sequence of rhodanese domain of P13_0027 for comparison with other rhodanese domains in other organisms
EIEIQHSAKKVDCNKRGISVFYCD

SEQ ID NO: 4 - protein, 47 aa, artificial sequence
  Amino acid sequence for PTP domain of P13_0027 for comparison with other PTP domains
VPGLGKQNMNNQNNVLVHCEAGIKESRSSISPTICMAYLMKKLPRLE

SEQ ID NO: 5 - DNA, 312 bp, artificial sequence
  Nucleic acid sequence for rhodanese domain of P13_0027
ttacatacaactattattaaatgaaggaagtaacaaaagtgtatacatattagatattcgaaaagaagatttatttaaccagggtcatattaagagtagtattaatatatataataaaaagatgatgatacaaataaataaagaaatgatatgtaaagacaatttaaaaattatcttttatgatcagaataatatgaataatatatatgatgattgtattaatttatataatgtctatttttcaaatattaaagtagagaacatatacattttaaaaggaggatatgaagattttgaaagagaatattgttt

SEQ ID NO: 6 - DNA, 705 bp, artificial sequence
  DNA sequence of PTP domain of P13_0027
aaattatcctataaaaatgtttgataacttatatttaggaaacattattcatataaataatatttttataaatgattttttgaacatcaaatatatttatgattttacatcaactggttttgttataaaaacagaaaataaagaaacaaggaaaaataaggaacttttatattttagatataatgtatatactaaaaattttgaaaatacaaattctttgaataatgaatctattaattattacaattttttagatatacatatgatatataaagttattacttctatgataagtacaaatcaaaatgatatatctcataataataataataatgatgatgatgatgtaatatataatgatcagaataataatatggctttatgtacaaaccaagtaaaagataataatacatcatttataaaacaaaataaagaacagttaatatgttatataaattctacacataataacaaaaaacaaaatatgaataatcaaaataatatccttattatatgtaatcatggaatgaaaaatcctacatcagaaaaaacaaatagtataagtcttattatatgtatgtgttatattatgtatataaaaaaatataaccctaatttaattattgcctatatgctaaaaatatataataactggagtataaattctcagacaaaatcatttttagaaagtttctatcaatccct

SEQ ID NO: 7 - protein, 22 aa, Homo sapiens
ELIQHSAKKVDIDCSQKVVVYD

SEQ ID NO: 8 - protein, 22 aa, Pan troglodytes
ELIQHSAKKVDIDCSQKVVVYD

SEQ ID NO: 9 - protein, 17 aa, Macaca mulatta
PIVPLDTQKRIIIVFCE

SEQ ID NO: 10 - protein, 17 aa, Canis Familiaris
PIVPLDTQKRIIIVFCE

SEQ ID NO: 11 - protein, 22 aa, Mus musculus
ELIQHSAKKVDIDCNQKVVVYD

SEQ ID NO: 12 - protein, 15 aa, Hydra magnipapillata
PKIKNGKRIVLVFCE

SEQ ID NO: 13 - protein, 18 aa, Trichomonas vaginalis
LVNDCAKDGISTLVFYCQ

SEQ ID NO: 14 - protein, 15 aa, Plasmodium vivax
EIELKQNAKVIFYYT

SEQ ID NO: 15 - protein, 16 aa, Plasmodium chabaudi chabaudi
EIIKSKNENIKIIFYD

SEQ ID NO: 16 - protein, 16 aa, Plasmodium yoeli yoeli
EIIKKKNENIKIIFYD

SEQ ID NO: 17 - protein, 14 aa, Plasmodium knowlesi
EIELKKDAKVVFYA

SEQ ID NO: 18 - protein, 16 aa, Plasmodium berghei
DIIKKKNENIKIIFYD

SEQ ID NO: 19 - protein, 14 aa, Plasmodium falciparum
EMICKDNLKIIFYD

SEQ ID NO: 20 - protein, 54 aa, Plasmodium falciparum
STHNNKKQNMNNQNNILIICNHGMKNPTSEKTNSISLIICMCYIMYIKKYNPNL

SEQ ID NO: 21 - protein, 51 aa, Plasmodium yoeli
NKHQPNNLKKNILIICNDGIKDESNKQKMNSISLIISMCYIIYSKKYNLNL

SEQ ID NO: 22 - protein, 19 aa, Plasmodium vivax
TKKCGPNLTIAYVLRINNN

SEQ ID NO: 23 - protein, 37 aa, Xenopus laevis
VKRAGGRVLVHCEAGISRSPTICMAYLMKTRKFHLEE

SEQ ID NO: 24 - protein, 37 aa, Penicillium marneffei
GKADRSATLVHCRVGVSRSATICIAEVMSSLNLSFPR

SEQ ID NO: 25 - protein, 37 aa, Mus musculus
VREEGGKVLVHCEAGVSRSPTICMAYLMKTKQFRLKE

SEQ ID NO: 26 - protein, 37 aa, Rattus norvegicus; identical to SEQ ID NO: 25

SEQ ID NO: 27 - protein, 37 aa, Gallus galus
VRRAGGKILVHCEAGISRSPTICMAYLMKTKKLRLEE

SEQ ID NO: 28 - protein, 37 aa, Homo sapiens
VREKGGKVLVHCEAGISRSPTICMAYLMKTKQFRLKE

SEQ ID NO: 29 - protein, 37 aa, Aspergillus flavus
GKKDGTATLVHCRVGVSRSATICIAEVMASLGLSFPR

SEQ ID NO: 30 - protein, 37 aa, Pan trogodytes; identical to SEQ ID NO: 28

SEQ ID NO: 31 - protein, 37 aa, Canis familiaris
VREKGGKVLVHCEAGISRSPTICMAYLMKAKQFRLKD

SEQ ID NO: 32 - protein, 37 aa, Danio rerio
VKAEGGKVLVHCEAGISRSPTICMAYIMKTQRLRLEQ

SEQ ID NO: 33 - protein, 37 aa, Nematostella vectensis
VKASGGRVLVHCQAGISRSATICLAYLISRLNFRLDE

SEQ ID NO: 34 - protein, 37 aa, Leishmania braziliensis
ARSHKKGILIHCFAGLSRSVTIAVAYLMHLKGIPRDE

SEQ ID NO: 35 - DNA, 45 bp, artificial sequence
  Forward primer for the P13_0027 phosphatase domain
aaaggatccatgtatataaattatcctataaaaatgtttgataac

SEQ ID NO: 36 - DNA, 36 bp, artificial sequence
  Reverse primer for the P13_0027 phosphatase domain
tttgaattccttaattagggattgatagaaactttc